(19) United States
(12) Patent Application Publication
(10) Pub. No.: US 2026/0200945 A1
(43) Pub. Date: Jul. 16, 2026

(54) 4-(3,8-DIAZABICYCLO[3,2,1]OCTAN-3-YL)-PYRIDO[4,3-D]PYRIMIDINE DERIVATIVES AS INHIBITORS OF THE KRAS (G12D) MUTANT ONCOPROTEIN FOR THE TREATMENT OF CANCER

(71) Applicant: Ranok Therapeutics (Hangzhou) Co. Ltd., Hangzhou (CN)

(72) Inventors: Weiwen Ying, Lexington, MA (US); Chenghao Ying, Shanghai (CN); Kevin P. Foley, Waltham, MA (US); Zhiyong Wang, Hangzhou, Zhejiang (CN); Wei Yin, Lianyungang (CN); Liang Ma, Lianyungang (CN); Guoqiang Wang, Lexington, MA (US); Jinhua Li, Hangzhou, Zhejiang (CN); Yaya Wang, Hangzhou, Zhejiang (CN); Yan Dai, Hangzhou, Zhejiang (CN); Thomas Prince, Danville, PA (US)

(21) Appl. No.: 19/134,385

(22) PCT Filed: Nov. 30, 2023
(86) PCT No.: PCT/US2023/081835

§ 371 Date: May 30, 2025

(30) Foreign Application Priority Data
Dec. 1, 2022 (WO) PCT/CN2022/136034

(51) Int. Cl. C07D 519/00; A61K 31/519; A61K 31/55; A61P 35/00; C07B 59/00

(52) U.S. Cl. CPC C07D 519/00; A61K 31/519; A61K 31/55; A61P 35/00; C07B 59/002

(57) ABSTRACT

Provided are small molecule inhibitors of the KRAS(G12D) mutant oncoprotein having the structural formula (I) and pharmaceutically acceptable salts and compositions thereof, which are useful for treating cancers and related conditions.

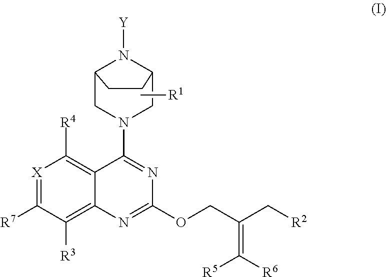

### RELATED APPLICATIONS

This application claims the benefit of priority to internal application No. PCT/CN2022/136034, filed Dec. 1, 2022, the entire contents of which are incorporated herein by reference.

### BACKGROUND

Discovered as a human oncogene in the early 1980s, the Kirsten rat sarcoma virus homolog (KRAS) gene encodes a monomeric small 21 kDa GTPase that has long been an elusive cancer drug target (Chang et al., PNAS, 1982, 79:4848-52; McCoy et al., Nature, 1983, 302:79-8). KRAS functions as a molecular switch for promoting cell growth by cycling between GTP- and GDP-bound states. In the GTP-bound state, KRAS signals for growth through the RAF-MAPK and PI3K-AKT-MTOR pathways. KRAS subsequently hydrolyzes GTP to GDP with the aid of GTPase activating proteins (GAPs). This GDP-bound state switches “off” KRAS pro-growth signaling. KRAS can then be switched back “on” by GDP to GTP exchange through the aid of guanine nucleotide exchange factors, such as SOS1 (Cox and Der, Small GTPases, 2010, 1:2-27; Kerk et al., Nat Rev Cancer, 2021, 21:510-525). Preventing this exchange by locking KRAS in the GDP-bound state is a practical method for inhibiting its growth promoting activity.

The human KRAS gene is encoded on Chromosome 12p12.1 and is among the most frequently mutated genes in human cancers (Pylayeva-Gupta et al., Nat Rev Cancer, 2011, 11:761-774). Mutations that prevent GTP-hydrolysis lock KRAS in the active GTP-bound state and reprogram cells for perpetual proliferation. KRAS mutated from glycine (G) at the 12th codon to aspartate (D) creates a chronically active KRAS(G12D) oncoprotein, the gene for which is observed in 6.8% of cancers cases as analyzed by next-generation sequencing (Zhou et al., Pathol Oncol Res, 2020, 26:2835-2837). In tumor type-specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-Löpez et al., World J Gastroenterol, 2017, 23(39):7087-709; Miglio et al., Pathol Res Pract, 2014, 210:307-11; Gou et al., Br J Cancer, 2020, 22:857-867), among other cancers. Historically, oncogenic KRAS mutants have been considered undruggable (McCormick F, Biochem J, 2019, 476:356-74), however the discovery of an allosteric pocket in GDP-bound KRAS has allowed the search for small molecule inhibitors (Ostrem et al., Nature, 2013, 503: 548-51). The G12D mutation moreover provides a unique chemical moiety-binding space due to the encoding of an acidic amino acid residue (D) in place of a small flexible amino acid residue possessing only a hydrogen side chain (G). This alteration of the KRAS protein structure provides a unique space that may be targeted with small molecules drugs that specifically inhibit KRAS(G12D) oncogenic activity. It is therefore desirable to design and develop small molecule drugs that target KRAS(G12D) with sufficient bioavailability to treat diseases such as cancer.

### SUMMARY

Provided herein are small molecule inhibitors of the KRAS(G12D) mutant oncoprotein. Inhibitors of KRAS(G12D) include those having the structural formula I:

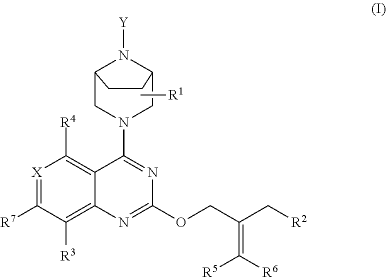

and pharmaceutically acceptable salts and compositions comprising such, wherein Y, R¹, R², R³, R⁴, R⁵, and R⁶ are as defined herein. The use of these compounds, salts, and compositions for treating diseases responsive to the inhibition of KRAS(G12D), such as cancer, is also disclosed.

### DETAILED DESCRIPTION

### 1. General Description of Compounds

As part of a first embodiment, provided is a compound of the Formula I:

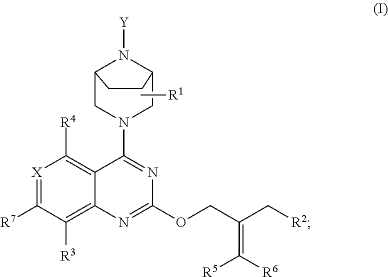

or a pharmaceutically acceptable salt thereof, wherein
   Y is hydrogen or —C(O)OCHR^{a}OC(O)R^{b};
   X is —CR^{4a} or N;
   R¹ is hydrogen, halo, OH, (C₁-C₄)alkyl, (C₁-C₄)haloalkyl, (C₁-C₄)hydroxyalkyl, —CHO, —C(O)OR^{b}, —C(O)ONR^{a}R^{b} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;
   R² is a heterocyclyl optionally substituted with 1 to 3 groups selected from R¹;
   R³ is selected from hydrogen, halo, (C₁-C₄)alkyl, cyano, and (C₃-C₆)cycloalkyl optionally substituted with 1 to 3 groups selected from halo, (C₁-C₄)alkyl, (C₁-C₄)alkoxy, halo(C₁-C₄)alkoxy, and cyano;
   R⁴ is selected from hydrogen, (C₁-C₄)alkyl, (C₁-C₄)haloalkyl, (C₁-C₄)alkoxy, deuterated (C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
   R^{4a} is selected from hydrogen, halo, (C₁-C₄)alkyl, (C₁-C₄)haloalkyl, C₁-C₄)haloalkoxy, (C₂-C₄)alkynyl, (C₂-C₄)alkenyl, (C₁-C₄)alkoxy, halo(C₁-C₄)alkoxy, (C₃-C₆)cycloalkyl, —O(C₃-C₆)cycloalkyl, cyano, NH₂, —NH(C₁-C₄)alkyl, and —N[(C₁-C₄)alkyl]₂; or R⁴ and R^{4a} are taken together with the atoms to which they are respectively attached form a 5- to 6-membered heterocyclyl ring or a 5- to 6-membered heteroaryl ring, each optionally substituted with 1 or 2 groups selected from halo, (C₁-C₄)alkyl, (C₁-C₄)haloalkyl, (C₁-C₄)alkoxy, and (C₁-C₄)haloalkoxy;
   R⁵ and R⁶ are each independently selected from hydrogen, halo, (C₁-C₄)alkyl, (C₁-C₄)haloalkyl, (C₁-C₄)alkoxy, deuterated (C₁-C₄)alkoxy, and (C₁-C₄)haloalkoxy; or R⁵ and R⁶ are taken together with the carbon they are attached to form (C₃-C₆)cycloalkyl or 4- to 8-membered monocyclic heterocyclyl, wherein said (C₃-C₆)cycloalkyl and said 4- to 8-membered monocyclic heterocyclyl are optionally substituted with 1 to 3 groups selected from halo, (C₁-C₄)alkyl, (C₁-C₄)alkoxy, halo(C₁-C₄)alkoxy, and cyano;
   R⁷ is selected from aryl or 5- to 10-membered heteroaryl, wherein said aryl and 5- to 10-membered heteroaryl are each optionally substituted with one or more groups selected from R^{7a};
   R^{7a} is selected from halo, (C₁-C₄)alkyl, halo(C₁-C₄)alkyl, (C₁-C₄)alkoxy, halo(C₁-C₄)alkoxy, (C₂-C₄)alkenyl, (C₂-C₄)alkynyl, cyano, OH, NH₂, —NH(C₁-C₄)alkyl, —N[(C₁-C₄)alkyl]₂, (C₃-C₆)cycloalkyl, and —O(C₃-C₆)cycloalkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
R^{a} and R^{b} are each independently selected from hydrogen and (C₁-C₄)alkyl; and
R^{c} is selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO₂.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein the term “comprising” or “comprises” are used in reference to compositions, methods, and respective component(s) thereof, that are present in a given embodiment, yet open to the inclusion of unspecified elements.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from, unless otherwise specified, 3 to 10 carbon atoms (e.g., from 3 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group=O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“deuterated alkoxy” refers to an alkoxy group in which one or more hydrogens (e.g., one or two hydrogens) has been replaced by deuterium.

“Haloalkoxy” is a haloalkyl group which is attached to another moiety via an oxygen atom such as, e.g., —OCHF₂ or —OCF₃.

As used herein, the term “halogen” or “halo” means F, Cl, Br or I.

The term “aryl” refers to monocyclic and bicyclic carbon ring systems having a total of six to 10 ring members, wherein at least one ring in the system is aromatic. Examples include, but are not limited to phenyl, naphthyl, anthracyl and the like. It will be understood that when specified, optional substituents on an aryl group may be present on any substitutable position.

As used herein, the term “heterocyclyl” means a 4- to 12-membered monocyclic or polycyclic (e.g., a bridged, fused, or spiro bicyclic ring) saturated or partially unsaturated heterocyclic ring containing 1 to 4 heteroatoms independently selected from N, O, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, 2-azaspiro[3.3]heptanyl, 6-oxa-2-azaspiro[4.5]decanyl, 5-azaspiro[2.4]heptanyl 2,7-diazaspiro[3.5]nonanyl, 2-azaspiro[3.5]nonanyl, 3-azabicyclo[3.1.0]hexanyl, 2-azabicyclo[3.1.0]hexanyl, 8-azabicyclo[3.2.1]octanyl, 3,8-diazabicyclo[3.2.1]octanyl, 3,6-diazabicyclo[3.1.1]heptanyl, octahydrocyclopenta[c]pyrrolyl, octahydro-1H-pyrrolo[2,3-c]pyridinyl, 1,4-dioxa-8-azaspiro[4.5]decanyl, 2-oxa-6-azaspiro[3.3]heptanyl, 6-oxa-2-azaspiro[3.4]octanyl, 8-oxa-2-azaspiro[4.5]decanyl, 4,5,6,7-tetrahydrothieno[2,3-c]pyridinyl, 1-oxa-7-azaspiro[4.4]nonanyl, 6,7-dihydro-5H-pyrrolo[3,4-b]pyridinyl, or 1,2,3,6-tetrahydropyridinyl and the like. Optional substituents on a heterocyclyl group may be present on any substitutable position and, include, e.g., the position at which the heterocyclyl is attached, valence permitting.

The term “spiro” refers to two rings that shares one ring atom (e.g., carbon).

The term “fused” refers to two rings that share two adjacent ring atoms with one another.

The term “bridged” refers to two rings that share three ring atoms with one another.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached, valence permitting.

When used in connection to describe a chemical group that may have multiple points of attachment, a hyphen (-) designates the point of attachment of that group to the variable to which it is defined. For example, —(C₁-C₄)alkylaryl and means that the point of attachment for these groups occurs on the alkyl group.

A hash bond as in “ [symbol] ” represents the point at which the depicted group is attached to the defined variable.

The term “KRAS” refers to the protein product of the KRAS proto-oncogene, GTPase gene.

The term “KRAS(G12D)” refers to the protein product of the KRAS gene carrying a mutation that results in the glycine amino acid at position 12 of KRAS being replaced by an aspartate.

A “chemical entity which binds KRAS^{G12D}” refers to a small molecule or a distinct portion of a larger molecule which binds to a portion of KRAS^{G12D}. In some aspects, the chemical entity which binds KRAS^{G12D} is a small molecule. In some aspects, the chemical entity which binds KRAS^{G12D} is a small molecule having a molecular weight of less than 2,000 g/mol. In some aspects, the chemical entity which binds KRAS^{G12D} induces a confirmation change in KRAS^{G12D}.

The term “SOS1” refers to the protein product of the SOS1 gene that functions as a guanine nucleotide exchange factor for RAS proteins.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments, an agent is administered intravenously, intramuscularly, subcutaneously, intradermally, intranasally, orally, transcutaneously, or mucosally. In certain embodiments, an agent is administered intravenously. In In certain embodiments, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

As part of a second embodiment, Y in the compound of Formula I is hydrogen, wherein the remaining variables are as described above in the first embodiment.

As part of a third embodiment, R¹ in the compound of Formula I is hydrogen, wherein the remaining variables are as described above in the first or second embodiment.

As part of a fourth embodiment, R⁴ in the compound of Formula I is hydrogen or (C₁-C₄)alkoxy, wherein the remaining variables are as described above in any one of the first to third embodiments. Alternatively, as part of a fourth embodiment, R⁴ in the compound of Formula I is OCH₃, wherein the remaining variables are as described above in any one of the first to third embodiments.

As part of a fifth embodiment, X in the compound of Formula I is N, wherein the remaining variables are as described above in any one of the first to fourth embodiments. Alternatively, as part of a fifth embodiment, X in the compound of Formula I is —CR^{4a} and R⁴ and R^{4a} are taken together with the atoms to which they are respectively attached form a 5-membered heterocyclyl ring, wherein the remaining variables are as described above in any one of the first to fourth embodiments. In another alternative, as part of a fifth embodiment, X in the compound of Formula I is —CR^{4a} and R⁴ and R^{4a} are taken together with the atoms to which they are respectively attached form a dihydrofuranyl or a dihydropyrazolyl ring, wherein the remaining variables are as described above in any one of the first to fourth embodiments.

As part of a sixth embodiment, R³ in the compound of Formula I is halo, wherein the remaining variables are as described above in any one of the first to fifth embodiments. Alternatively, part of a sixth embodiment, R³ in the compound of Formula I is fluoro, wherein the remaining variables are as described above in any one of the first to fifth embodiments.

As part of a seventh embodiment, R⁵ and R⁶ in the compound of Formula I are each independently selected from hydrogen, halo, (C₁-C₄)alkyl, and (C₁-C₄)haloalkyl, wherein the remaining variables are as described above in any one of the first to sixth embodiments. Alternatively, part of a seventh embodiment, R⁵ and R⁶ in the compound of Formula I correspond to structures selected from

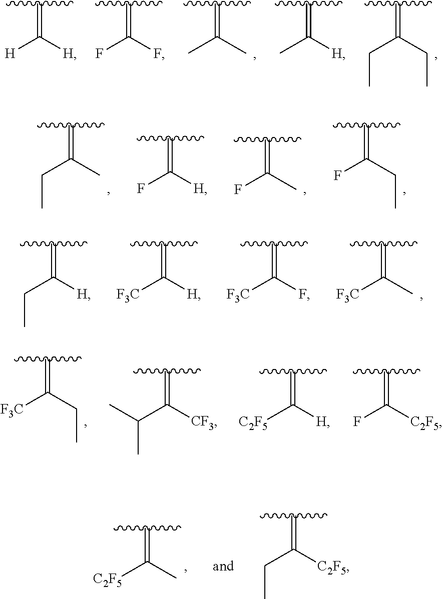

wherein the remaining variables are as described above in any one of the first to sixth embodiments. Alternatively, part of a seventh embodiment, R⁵ and R⁶ in the compound of Formula I correspond to structures selected from

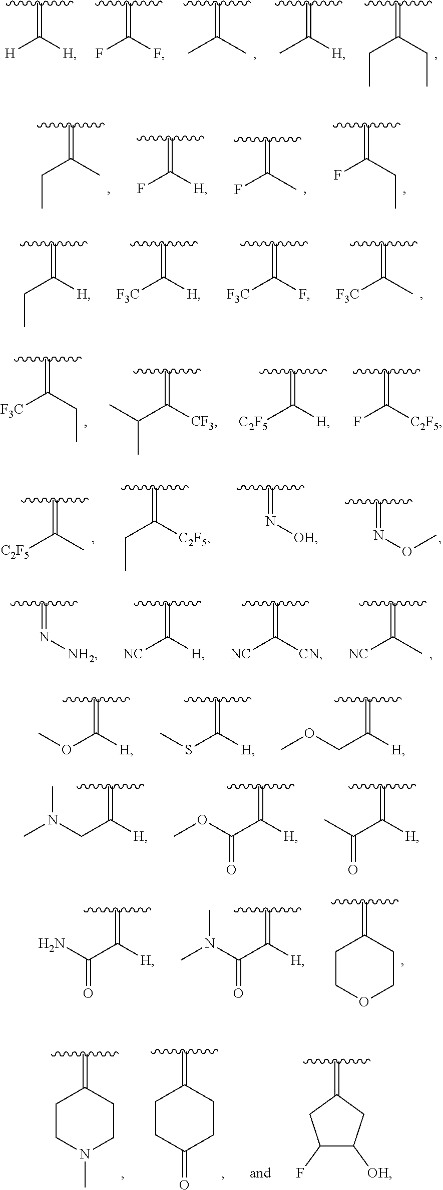

wherein the remaining variables are as described above in any one of the first to sixth embodiments. In another alternative, as part of a seventh embodiment, R⁵ and R⁶ in the compound of Formula I are taken together with the carbon they are attached to form (C₃-C₆)cycloalkyl or 4- to 6-membered monocyclic heterocyclyl, each optionally substituted with 1 to 3 groups selected from halo, (C₁-C₄)alkyl, (C₁-C₄)alkoxy, halo(C₁-C₄)alkoxy, and cyano, wherein the remaining variables are as described above in any one of the first to sixth embodiments. In another alternative, as part of a seventh embodiment, R⁵ and R⁶ in the compound of Formula I are taken together with the carbon they are attached to form cyclopropyl, cyclobutyl, cyclopentyl, cyclohexyl, oxetanyl, azetidinyl, tetrahydropyranyl, or piperidinyl, each optionally substituted with 1 to 3 (C₁-C₄)alkyl, wherein the remaining variables are as described above in any one of the first to sixth embodiments.

As part of an eighth embodiment, R⁷ in the compound of Formula I is selected from phenyl, naphthalenyl, 6-membered heteroaryl, 9-membered heteroaryl, or 10-membered heteroaryl, each of which are optionally substituted with one or more groups selected from R^{7a}, wherein the remaining variables are as described above in any one of the first to seventh embodiments. Alternatively, as part of an eighth embodiment, R⁷ in the compound of Formula I is selected from phenyl, naphthalenyl, pyridinyl, benzothiazolyl, or isoquinolinyl, each of which are optionally substituted with one or more groups selected from R^{7a}, wherein the remaining variables are as described above in any one of the first to seventh embodiments.

As part of a ninth embodiment, R^{7a} in the compound of Formula I is selected from halo, (C₁-C₄)alkyl, halo(C₁-C₄)alkyl, halo(C₁-C₄)alkoxy, (C₂-C₄)alkynyl, cyano, OH, NH₂, and (C₃-C₆)cycloalkyl, wherein the remaining variables are as described above in any one of the first to eighth embodiments. Alternatively, as part of a ninth embodiment, R^{7a} in the compound of Formula I is selected from fluoro, chloro, ethyl, ethynyl, CF₃, C₂F₅, cyano, OCF₃, OH, NH₂, and cyclopropyl, wherein the remaining variables are as described above in any one of the first to eighth embodiments.

As part of a tenth embodiment, R⁷ in the compound of Formula I is selected from

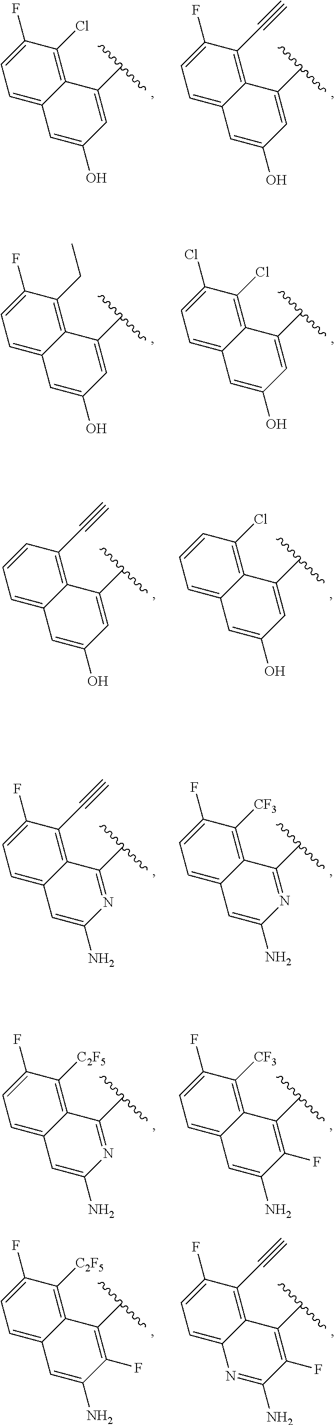

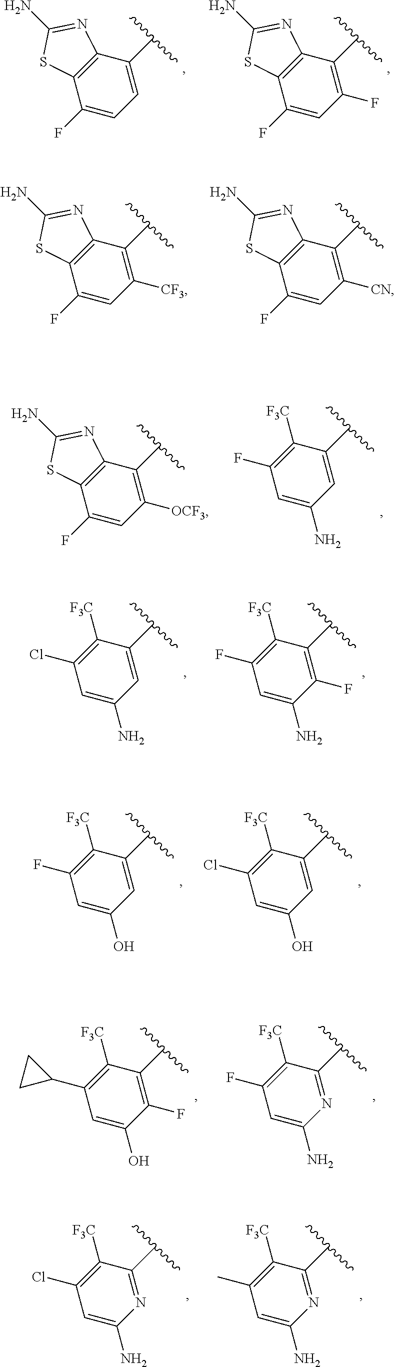

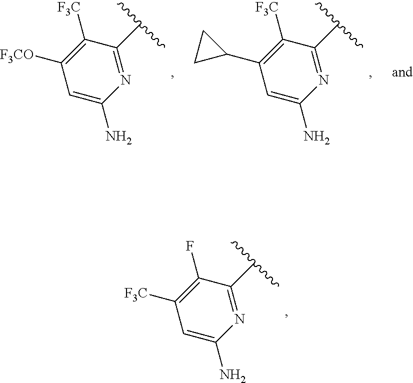 [SMILES: *c1nc(N)cc(OC(F)(F)F)c1C(F)(F)F ; *c1nc(N)cc(C2CC2)c1C(F)(F)F ; *c1nc(N)cc(C(F)(F)F)c1F]

wherein the remaining variables are as described above in any one of the first to ninth embodiments. Alternatively, as part of a tenth embodiment, R⁷ in the compound of Formula I is selected from

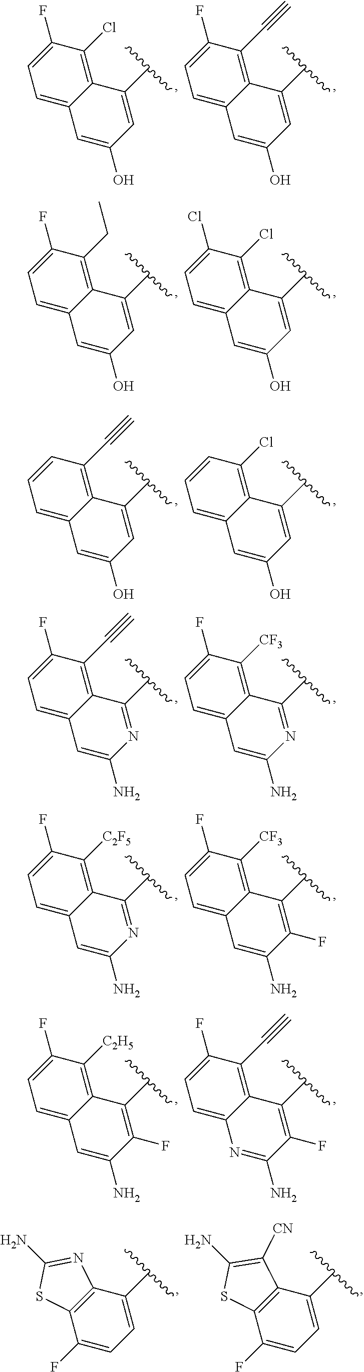

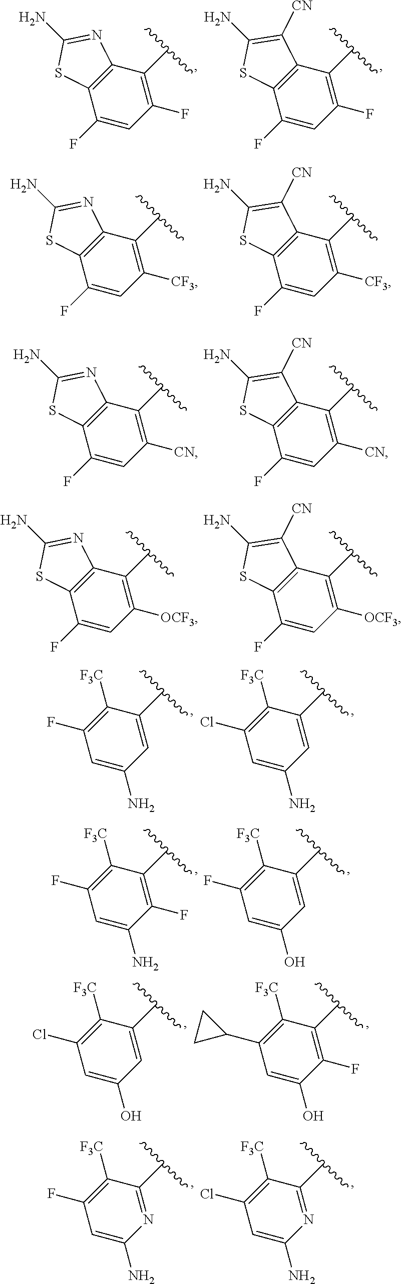

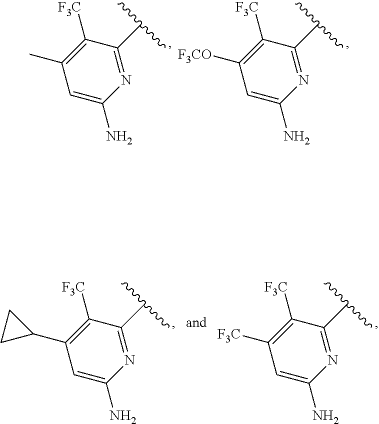 [SMILES: *c1nc(N)cc(C)c1C(F)(F)F ; *c1nc(N)cc(OC(F)(F)F)c1C(F)(F)F ; *c1nc(N)cc(C2CC2)c1C(F)(F)F ; *c1nc(N)cc(C(F)(F)F)c1C(F)(F)F]

wherein the remaining variables are as described above in any one of the first to ninth embodiments.

As part of an eleventh embodiment, R² in the compound of Formula I is a 4- to 6-membered monocyclic heterocyclyl or a 6- to 10-membered bicyclic heterocyclyl, each optionally substituted with 1 to 3 groups selected from R^{c}, wherein the remaining variables are as described above in any one of the first to tenth embodiments. Alternatively, as part of an eleventh embodiment, R² in the compound of Formula I is pyrrolidinyl, 3-azabicyclo[3.1.0]hexanyl, octahydrocyclopenta[c]pyrrolyl, 6-oxa-2-azaspiro[4.5]decanyl, 5-azaspiro[2.4]heptanyl, 2-azaspiro[3.3]heptanyl, 2-oxa-6-azaspiro[3.3]heptanyl, 6-oxa-2-azaspiro[3.4]octanyl, 8-oxa-2-azaspiro[4.5]decanyl, azetidinyl, piperidinyl, morpholinyl, piperizinyl, 6-azaspiro[2.5]octanyl, 8-azabicyclo[3.2.1]octanyl, 3-oxa-8-azabicyclo[3.2.1]octanyl, 5,6,7,8-tetrahydro-1,7-naphthyridinyl, 3-oxa-8-azabicyclo[3.2.1]octanyl, 4,5,6,7-tetrahydrothieno[2,3-c]pyridinyl, 2-oxa-7-azaspiro[3.5]nonanyl, 2-oxa-7-azaspiro[4.4]nonanyl, 1-oxa-7-azaspiro[4.4]nonanyl, isoindolinyl, and 6,7-dihydro-5H-pyrrolo[3,4-b]pyridinyl, each optionally substituted with 1 to 3 groups selected from R^{c}, each optionally substituted with 1 to 3 groups selected from R^{c}, wherein the remaining variables are as described above in any one of the first to tenth embodiments.

As part of a twelfth embodiment, R^{c} in the compound of Formula I is selected from halo, (C₁-C₄)haloalkyl, (C₁-C₄)haloalkoxy, cyano, and —C(O)OR^{a}, wherein the remaining variables are as described above in any one of the first to eleventh embodiments.

As part of a thirteenth embodiment, R² is selected from

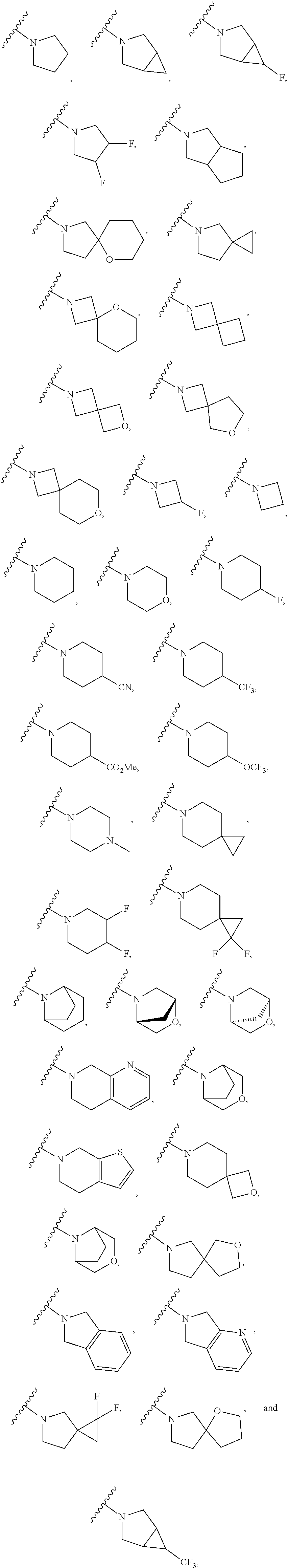

wherein the remaining variables are as described above in any one of the first to twelfth embodiments.

Additional compounds are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as inhibitors of KRAS(G12D). Their mechanisms of action include, but are not limited to, inhibiting KRAS(G12D) and thereby impeding down-stream signals that may result in inhibition of cancer cell growth and/or induction of cancer cell death or other KRAS or KRAS(G12D) functions. In one aspect, the disclosed compounds effectuate the inhibition of KRAS(G12D).

Thus, provided herein are methods of treating conditions which are responsive to the inhibition of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the inhibition of KRAS(G12D). Further provided is the use of a compound or composition described herein for treating conditions which are responsive to the inhibition of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

The compounds claimed herein were prepared following the procedures outlined in the Scheme 1.

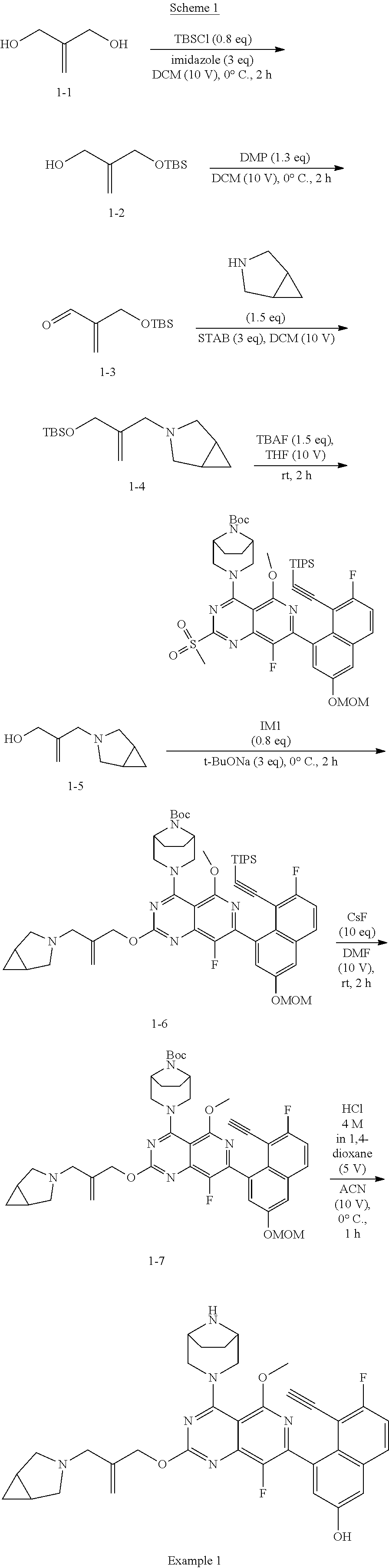

### Preparation of Example 1

Step 1: 2-{[(tert-butyldimethylsilyl)oxy]methyl}prop-2-en-1-ol. A solution of 2-methylidenepropane-1,3-diol (25 g, 283.749 mmol, 1 equiv) in DCM (250 mL) was treated with Imidazole (19.32 g, 283.749 mmol, 1 equiv) for 5 min at 0° C. under nitrogen atmosphere followed by the addition of TBSCl (34.21 g, 226.999 mmol, 0.8 equiv) dropwise at 0° C. The residue was purified by silica gel column chromatography, eluted with PE/EA (5:1) to afford 1-2 as a colorless oil. ¹H NMR (400 MHz, DMSO-d6) δ 4.95 (h, J=1.3 Hz, 2H), 4.70 (t, J=5.6 Hz, 1H), 4.06 (d, J=1.4 Hz, 2H), 3.86 (d, J=5.5 Hz, 2H), 0.83 (s, 9H). LCMS: (ES, m/z): 203 [M+H]⁺.

Step 2: 2-{[(tert-butyldimethylsilyl)oxy]methyl}prop-2-enal. A solution of intermediate 1-2 (5 g, 24.707 mmol, 1 equiv) and DMP (31.44 g, 74.121 mmol, 3 equiv) in DCM (50 mL) was stirred for 2 h at 0° C. under nitrogen atmosphere. The reaction was quenched with Water at 0° C. The resulting mixture was concentrated under vacuum. The crude product was used in the next step directly without further purification. LCMS: (ES, m/z): 201 [M+H]⁺.

Step 3: 3-(2-{[(tert-butyldimethylsilyl)oxy]methyl}prop-2-en-1-yl)-3-azabicyclo[3.1.0]hexane. A solution of intermediate 1-3 (4 g, 19.965 mmol, 1 equiv) in DCM (40 mL) was treated with 3-azabicyclo[3.1.0]hexane (2.49 g, 29.947 mmol, 1.5 equiv) for 30 min at room temperature under nitrogen atmosphere followed by the addition of STAB (12.69 g, 59.895 mmol, 3 equiv) dropwise at room temperature. The residue was purified by silica gel column chromatography, eluted with PE/EA (1:1) to afford 1-4 (1.5 g, 28.09%) as a light yellow oil. LCMS: (ES, m/z): 268 [M+H]⁺.

Step 4: 2-{3-azabicyclo[3.1.0]hexan-3-ylmethyl}prop-2-en-1-ol. A solution of intermediate 1-4 (400 mg, 1.495 mmol, 1 equiv) and TBAF (586.48 mg, 2.243 mmol, 1.5 equiv) in THF (4 mL) was stirred for 2 h at room temperature under nitrogen atmosphere. The resulting mixture was washed with 5×5 mL of water. The resulting mixture was concentrated under reduced pressure. The crude product was used in the next step directly without further purification. LCMS: (ES, m/z): 154 [M+H]⁺.

Step 5: tert-butyl 3-{2-[(2-{3-azabicyclo[3.1.0]hexan-3-ylmethyl}prop-2-en-1-yl)oxy]-8-fluoro-7-[7-fluoro-3-(methoxymethoxy)-8-[2-(triisopropylsilyl)ethynyl]naphthalen-1-yl]-5-methoxypyrido[4,3-d]pyrimidin-4-yl}-3,8-diazabicyclo[3.2.1]octane-8-carboxylate. A solution of intermediate 1-5 (200 mg, 1.305 mmol, 1 equiv) in was treated with t-BuONa (376.33 mg, 3.915 mmol, 3 equiv) for 30 min at 0° C. under nitrogen atmosphere followed by the addition of tert-butyl 3-{8-fluoro-7-[7-fluoro-3-(methoxymethoxy)-8-[2-(triisopropylsilyl)ethynyl]naphthalen-1-yl]-2-methanesulfonyl-5-methoxypyrido[4,3-d]pyrimidin-4-yl}-3,8-diazabicyclo[3.2.1]octane-8-carboxylate (333.66 mg, 0.391 mmol, 0.3 equiv) dropwise at 0° C. The resulting mixture was stirred for 1 h at room temperature under nitrogen atmosphere. The residue was purified by silica gel column chromatography, eluted with PE/EA (1:1) to afford 1-6 (300 mg, 24.84%) as a yellow solid. LCMS: (ES, m/z): 925 [M+H]⁺.

Step 6: tert-butyl 3-(8-fluoro-7-(7-fluoro-3-(methoxymethoxy)-8-((triisopropylsilyl)ethynyl)naphthalen-1-yl)-2-((1-((4-fluoropiperidin-1-yl)methyl)cyclopropyl)methoxy)pyrido[4,3-d]pyrimidin-4-yl)-3,8-diazabicyclo[3.2.1]octane-8-carboxylate. A solution of intermediate 1-6 (300 mg, 0.324 mmol, 1 equiv) and CsF (492.55 mg, 3.240 mmol, 10 equiv) in DMF (3 mL) was stirred for 1 h at room temperature under nitrogen atmosphere. The reaction was quenched with Water at room temperature. The aqueous layer was extracted with EtOAc (3×10 mL). The resulting mixture was concentrated under vacuum. The crude product was used in the next step directly without further purification. LCMS: (ES, m/z): 769 [M+H]⁺.

Step 7: 4-{2-[(2-{3-azabicyclo[3.1.0]hexan-3-ylmethyl}prop-2-en-1-yl)oxy]-4-{3,8-diazabicyclo[3.2.1]octan-3-yl}-8-fluoro-5-methoxypyrido[4,3-d]pyrimidin-7-yl}-5-ethynyl-6-fluoronaphthalen-2-ol. A solution of intermediate 1-7 (115 mg, 0.150 mmol, 1 equiv) and HCl(gas) in 1,4-dioxane (1 mL, 32.913 mmol, 220.05 equiv) in ACN (1 mL, 19.024 mmol, 127.19 equiv) was stirred for 1 h at 0° C. under nitrogen atmosphere. The mixture was neutralized to pH 7 with NH₃—H₂O. The resulting mixture was concentrated under vacuum. The crude product was purified by Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃) and ACN (35% ACN up to 75% in 10 min); Detector, uv 254 nm. The Example 1 was obtained as a white solid. ¹H NMR (400 MHz, DMSO-d6) δ 10.14 (s, 1H), 7.97 (dd, J=9.2, 5.9 Hz, 1H), 7.46 (t, J=9.0 Hz, 1H), 7.38 (d, J=2.5 Hz, 1H), 7.22 (d, J=2.5 Hz, 1H), 5.12-5.06 (m, 2H), 4.85 (s, 2H), 4.13-4.05 (m, 1H), 3.87 (d, J=5.8 Hz, 5H), 3.47 (s, 2H), 3.44-3.36 (m, 2H), 3.10 (s, 2H), 2.89 (d, J=8.6 Hz, 2H), 2.26-2.18 (m, 2H), 2.07 (s, 1H), 1.66-1.48 (m, 4H), 1.39-1.29 (m, 2H), 0.58 (q, J=3.7 Hz, 1H), 0.28 (td, J=7.7, 3.8 Hz, 1H). LCMS: (ES, m/z): 625 [M+H]⁺.

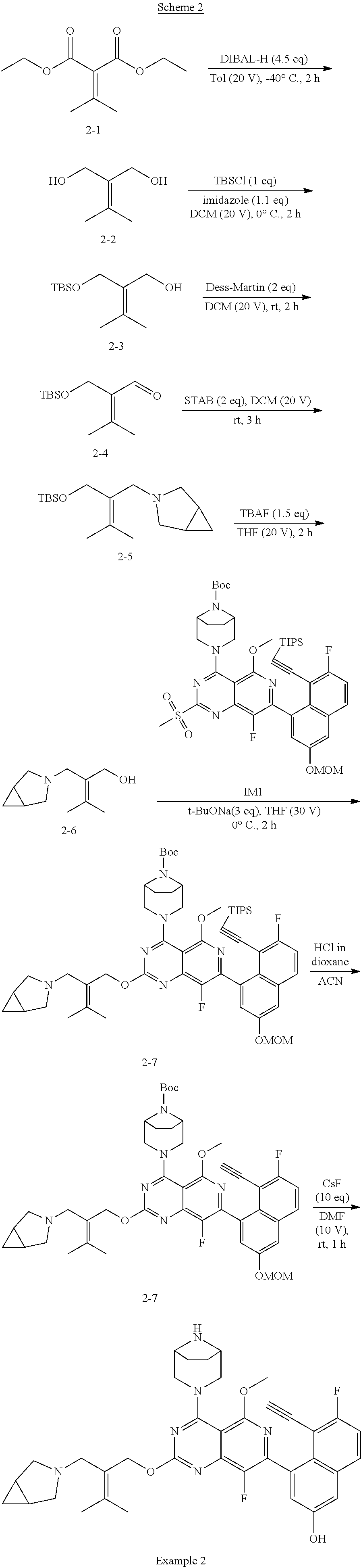

### Preparation of Example 2

Step 1: 2-(propan-2-ylidene)propane-1,3-diol. To a stirred solution of 1,3-diethyl 2-(propan-2-ylidene)propanedioate (5 g, 24.971 mmol, 1 equiv) in Toluene (100 mL) was added DIBAl-H (112.37 mL, 112.370 mmol, 4.5 equiv) dropwise at −40° C. under nitrogen atmosphere. The resulting mixture was stirred for 3 h at −40° C. under nitrogen atmosphere. The reaction was quenched by the addition of MeOH (20 mL) at −40° C. The resulting mixture was diluted with water (100 mL). The resulting mixture was extracted with EtOAc (3×100 mL). The combined organic layers were dried over anhydrous Na₂SO₄. After filtration, the filtrate was concentrated under reduced pressure. The crude product (2 g) was used in the next step directly without further purification. The LC-MS shows no MS signals. ¹H NMR (400 MHz, Chloroform-d) δ 4.30 (s, 4H), 2.26 (s, 2H), 1.77 (s, 6H).

Step 2: 2-{[(tert-butyldimethylsilyl)oxy]methyl}-3-methylbut-2-en-1-ol. To a stirred solution of 1-2 (2 g, 17.218 mmol, 1 equiv) and Imidazole (1.29 g, 18.940 mmol, 1.1 equiv) in DCM (20 mL) was added TBSCl (2.60 g, 17.218 mmol, 1 equiv) dropwise at 0° C. under nitrogen atmosphere. The resulting mixture was stirred for 2 h at room temperature under nitrogen atmosphere. The reaction was quenched with Water (50 mL) at room temperature. The resulting mixture was extracted with CH₂Cl₂ (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (10:1) to afford intermediate 1-3 (600 mg, 15.12%) as a colorless oil. The LC-MS shows no MS signals. ¹H NMR (400 MHz, Chloroform-d) δ 4.38-4.33 (m, 2H), 4.24 (s, 2H), 1.77 (d, J=1.1 Hz, 3H), 1.71 (s, 3H), 0.91 (d, J=0.8 Hz, 10H), 0.09 (s, 6H).

Step 3: 2-{[(tert-butyldimethylsilyl)oxy]methyl}-3-methylbut-2-enal. To a stirred solution of 1-3 (600 mg, 2.604 mmol, 1 equiv) in DCM (10 mL) was added Dess-Martin (2208.86 mg, 5.208 mmol, 2 equiv) at room temperature under nitrogen atmosphere. The resulting mixture was stirred for 2 h at room temperature under nitrogen atmosphere. The reaction was quenched with sat. NaHCO₃(aq.) (100 mL) and stirred 15 min at room temperature. The resulting mixture was extracted with CH₂Cl₂ (2×100 mL). The combined organic layers were washed with brine (1×100 mL), dried over anhydrous Na₂SO₄. After filtration, the filtrate was concentrated under reduced pressure. The crude product was used in the next step directly without further purification.

Step 4: 3-(2-{[(tert-butyldimethylsilyl)oxy]methyl}-3-methylbut-2-en-1-yl)-3-aza bicyclo[3.1.0]hexane. To a stirred solution of 1-4 (600 mg, 2.627 mmol, 1 equiv) and 3-aza bicyclo[3.1.0]hexane hydrochloride (471.22 mg, 3.940 mmol, 1.5 equiv) in DCM (10 mL) was added STAB (1113.49 mg, 5.254 mmol, 2.00 equiv) in portions at room temperature un der nitrogen atmosphere. The resulting mixture was stirred for 3 h at room temperature under nitrogen atmosphere. The reaction was quenched with Water (100 mL) at room temperature. The resulting mixture was extracted with CH₂Cl₂ (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (8:1) to afford intermediate 1-5 (250 mg, 32.20%) as a colorless oil.

Step 5: 2-{3-azabicyclo[3.1.0]hexan-3-ylmethyl}-3-methylbut-2-en-1-ol. To a stirred solution of 1-5 (250 mg, 0.846 mmol, 1 equiv) in THF (5 mL) was added TBAF (1.27 mL, 1.269 mmol, 1.5 equiv) at room temperature under nitrogen atmosphere. The resulting mixture was stirred for 1 h at room temperature under nitrogen atmosphere. The resulting mixture was diluted with EA (100 mL). The resulting mixture was washed with 4×50 mL of water, washed with brine (1×50 mL), dried over anhydrous Na₂SO₄. After filtration, the filtrate was concentrated under reduced pressure. This resulted in intermediate 1-6 (150 mg, 97.82%) as a colorless oil. LCMS: (ES, m/z): 182 [M+H]⁺.

Step 6: tert-butyl 3-{2-[(2-{3-azabicyclo[3.1.0]hexan-3-ylmethyl}-3-methylbut-2-en-1-yl)oxy]-8-fluoro-7-[7-fluoro-3-(methoxymethoxy)-8-[2-(triisopropylsilyl)ethynyl]naphthalen-1-yl]-5-methoxypyrido[4,3-d]pyrimidin-4-yl}-3,8-diazabicyclo[3.2.1]octane-8-carboxylate. To a stirred solution of 1-6 (160 mg, 0.883 mmol, 1 equiv) and tert-butyl 3-{8-fluoro-7-[7-fluoro-3-(methoxymethoxy)-8-[2-(triisopropylsilyl)ethynyl]naphthalen-1-yl]-2-methanesulfonyl-5-methoxypyrido[4,3-d]pyrimidin-4-yl}-3,8-diazabicyclo[3.2.1]octane-8-carboxylate (526.44 mg, 0.618 mmol, 0.7 equiv) in THF (5 mL) was added t-BuONa (254.47 mg, 2.649 mmol, 3 equiv) at 0° C. under nitrogen atmosphere. The resulting mixture was stirred for 2 h at 0° C. under nitrogen atmosphere. The residue was purified by reversed-phase flash chromatography with the following conditions: column, C₁₈ silica gel; mobile phase, MeOH in Water (10 mmol/L NH₄HCO₃), 50% to 100% gradient in 15 min; detector, UV 254 nm. This resulted in intermediate 1-7 (90 mg, 10.70%) as a yellow solid. LCMS: (ES, m/z): 953 [M+H]⁺.

Step 7: tert-butyl 3-{2-[(2-{3-azabicyclo[3.1.0]hexan-3-ylmethyl}-3-methylbut-2-en-1-yl)oxy]-7-[8-ethynyl-7-fluoro-3-(methoxymethoxy)naphthalen-1-yl]-8-fluoro-5-methoxypyrido[4,3-d]pyrimidin-4-yl}-3,8-diazabicyclo[3.2.1]octane-8-carboxylate. To a stirred solution of 1-7 (90 mg, 0.094 mmol, 1 equiv) in ACN (1 mL) was added HCl(gas) in 1,4-dioxane (1 mL, 32.913 mmol, 348.61 equiv) at 0° C. under nitrogen atmosphere. The resulting mixture was stirred for 1 h at 0° C. under nitrogen atmosphere. The mixture was basified to pH 9 with NH₃·H₂O. The resulting mixture was extracted with CH2Cl2 (2×20 mL). The combined organic layers were washed with brine (1×20 mL), dried over anhydrous Na₂SO₄. After filtration, the filtrate was concentrated under reduced pressure. This resulted in intermediate 1-8 (70 mg, 93.04%) as a yellow solid.

Step 8: 4-{2-[(2-{3-azabicyclo[3.1.0]hexan-3-ylmethyl}-3-methylbut-2-en-1-yl)oxy]-4-{3,8-diazabicyclo[3.2.1]octan-3-yl}-8-fluoro-5-methoxypyrido[4,3-d]pyrimidin-7-yl}-5-ethynyl-6-fluoronaphthalen-2-ol. To a stirred solution of 1-8 (70 mg, 0.088 mmol, 1 equiv) in DMF (1 mL) was added CsF (133.43 mg, 0.880 mmol, 10 equiv) at room temperature under nitrogen atmosphere. The resulting mixture was stirred for 1 h. The residue was purified by Prep-HPLC with the following conditions (Column: XBridge Prep OBD C18 Column, 30*150 mm, 5 m; Mobile Phase A: Water (1% HAC), Mobile Phase B: ACN; Flow rate: 60 mL/min; Gradient: 40% B to 65% B in 8 min; Wave Length: 220 nm; RT1(min): 6.82) to afford Example 2 (7.48 mg, 12.59%) as a white solid. ¹HNMR (400 MHz, DMSO-d₆ ppm) δ 10.13 (s, 1H), 7.97 (dd, J=9.2, 5.9 Hz, 1H), 7.46 (t, J=9.0 Hz, 1H), 7.38 (s, 1H), 7.22 (d, J=2.6 Hz, 1H), 4.88 (s, 2H), 4.11 (s, 1H), 3.87 (d, J=5.6 Hz, 5H), 3.47 (d, J=6.4 Hz, 2H), 3.40-3.37 (m, 2H), 3.12 (s, 2H), 2.82 (dd, J=8.4, 2.7 Hz, 2H), 2.26 (d, J=8.3 Hz, 2H), 1.78 (d, J=12.8 Hz, 6H), 1.60 (s, 4H), 1.29 (s, 2H), 0.59-0.45 (m, 1H), 0.22 (td, J=7.6, 3.6 Hz, 1H). LCMS: (ES, m/z): 653 [M+H]⁺.

The following compounds in Table 1 was prepared according to the methods described above using the appropriate starting materials.

TABLE 1

| | | LCMS: | |
|---|---|---|---|
| | | m/z | ¹H NMR |
| Ex. | Structure | [M + H]⁺ | (DMSO-d₆, 400 MHz) |
| 3 | 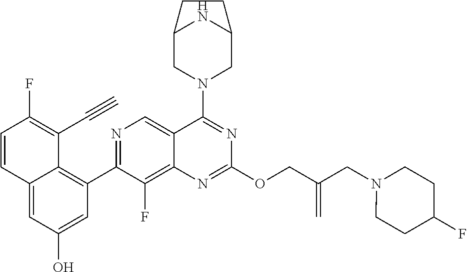 [SMILES: C#Cc1c(F)ccc2cc(O)cc(-c3ncc4c(N5CC6CCC(C5)N6)nc(OCC(=C)CN5CCC(F)CC5)nc4c3F)c12] | 615 | δ 10.42-10.02 (m, 1H), 9.07 (s, 1H), 8.05-7.90 (m, 1H), 7.47 (t, J = 9.0 Hz, 1H), 7.40 (s, 1H), 7.19 (s, 1H), 5.21 (s, 1H), 5.14 (s, 1H), 4.93 (s, 2H), 4.72 (d, J = 7.4 Hz, 1H), 4.64-4.51 (m, 1H), 4.45-4.35 (m, 1H), 3.91 (d, J = 6.2 Hz, 2H), 3.83- 3.76 (m, 2H), 3.65-3.57 (m, 2H), 3.02 (s, 2H), 2.48 (s, 1H), 2.28 (t, J = 9.3 Hz, 2H), 1.92- 1.74 (m, 6H), 1.74-1.59 (m, 2H). |
| 4 | 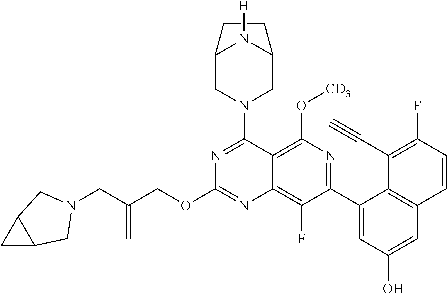 [SMILES: [2H]C([2H])([2H])Oc1nc(-c2cc(O)cc3ccc(F)c(C#C)c23)c(F)c2nc(OCC(=C)CN3CC4CC4C3)nc(N3CC4CCC(C3)N4)c12] | 628.3 | δ 8.23 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.5 Hz, 1H), 5.13- 5.05 (m, 2H), 4.85 (s, 2H), 4.21- 4.02 (m, 2H), 3.89 (s, 1H), 3.48 (s, 2H), 3.41 (s, 2H), 3.10 (s, 2H), 2.89 (d, J = 8.6 Hz, 2H), 2.23 (d, J = 8.5 Hz, 2H), 1.74-1.54 (m, 4H), 1.38-1.29 (m, 2H), 0.57 (q, J = 3.8 Hz, 1H), 0.28 (td, J = 7.7, 3.8 Hz, 1H) |
| 5 | 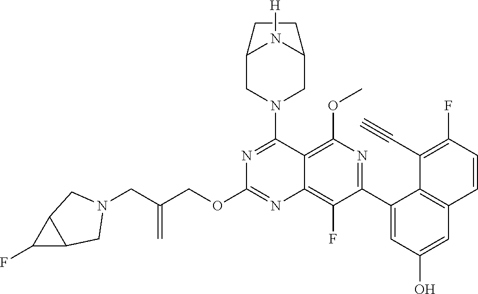 [SMILES: C#Cc1c(F)ccc2cc(O)cc(-c3nc(OC)c4c(N5CC6CCC(C5)N6)nc(OCC(=C)CN5CC6C(F)C6C5)nc4c3F)c12] | 643.1 | δ 8.25 (s, 1H), 7.97 (dd, J = 9.2, 5.9 Hz, 1H), 7.46 (t, J = 9.0 Hz, 1H), 7.39 (d, J = 2.5 Hz, 1H), 7.23 (d, J = 2.5 Hz, 1H), 5.15- 5.02 (m, 2H), 4.85 (s, 2H), 4.64 (d, J = 66.0 Hz, 1H), 4.20- 4.05 (m, 1H), 4.05-3.93 (m, 1H), 3.89 (s, 3H), 3.65-3.61 (m, 2H), 3.43-3.41 (m, 2H), 3.39-3.37 (m, 1H), 3.04 (s, 2H), 3.00 (dd, J = 8.9, 2.4 Hz, 2H), 2.35-2.25 (m, 2H), 1.79 (dd, J = 18.6, 2.2 Hz, 2H), 1.74- 1.54 (m, 4H) |
| 6 | 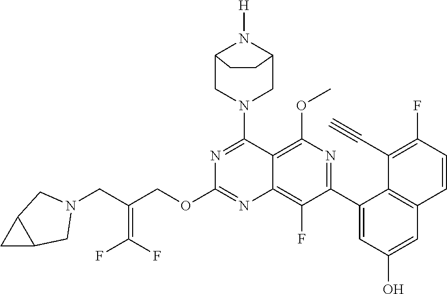 [SMILES: C#Cc1c(F)ccc2cc(O)cc(-c3nc(OC)c4c(N5CC6CCC(C5)N6)nc(OCC(CN5CC6CC6C5)=C(F)F)nc4c3F)c12] | 661.4 | 10.15 (s, 1H), 7.97 (dd, J = 9.2, 5.9 Hz, 1H), 7.47 (t, J = 9.0 Hz, 1H), 7.38 (d, J = 2.6 Hz, 1H), 7.22 (d, J = 2.6 Hz, 1H), 4.88 (s, 2H), 4.12 (s, 1H), 3.94- 3.54 (m, 5H), 3.51-3.43 (m, 2H), 3.42-3.38 (m, 2H), 3.11 (s, 2H), 2.87 (dd, J = 8.4, 2.0 Hz, 2H), 2.28 (d, J = 8.2 Hz, 2H), 1.65-1.42 (m, 4H), 1.36- 1.31 (m, 2H), 0.52 (q, J = 3.8 Hz, 1H), 0.26 (td, J = 7.7, 3.8 Hz, 1H) |
| 7 | 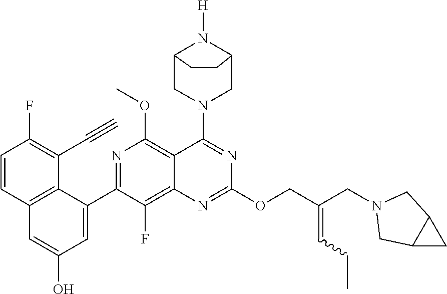 [SMILES: C#Cc1c(F)ccc2cc(O)cc(-c3nc(OC)c4c(N5CC6CCC(C5)N6)nc(OCC(=CCC)CN5CC6CC6C5)nc4c3F)c12] | 653.5 | |
| 8 | 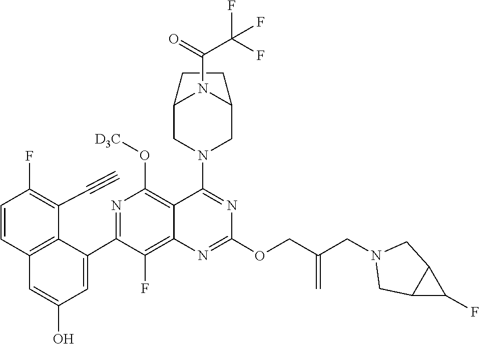 [SMILES: [2H]C([2H])([2H])Oc1nc(-c2cc(O)cc3ccc(F)c(C#C)c23)c(F)c2nc(OCC(=C)CN3CC4C(F)C4C3)nc(N3CC4CCC(C3)N4C(=O)C(F)(F)F)c12] | 742.1 | δ 10.17 (s, 1H), 7.98 (dd, J = 9.2, 5.9 Hz, 1H), 7.47 (t, J = 9.0 Hz, 1H), 7.39 (d, J = 2.6 Hz, 1H), 7.26-7.20 (m, 1H), 5.11 (d, J = 10.9 Hz, 2H), 4.87 (s, 2H), 4.80-4.50 (m, 3H), 4.45- 4.07 (m, 2H), 3.96 (d, J = 2.5 Hz, 1H), 3.57-3.43 (m, 2H), 3.05 (s, 2H), 3.00 (dd, J = 9.0, 2.9 Hz, 2H), 2.30 (d, J = 8.8 Hz, 2H), 1.95 (s, 1H), 1.86 (d, J = 12.7 Hz, 2H), 1.82-1.75 (m, 3H) |
| 9 | 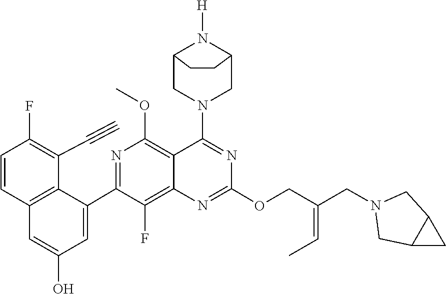 [SMILES: C#Cc1c(F)ccc2cc(O)cc(-c3nc(OC)c4c(N5CC6CCC(C5)N6)nc(OC/C(=C\C)CN5CC6CC6C5)nc4c3F)c12] | 639.5 | δ 10.19 (s, 1H), 8.21 (s, 1H), 7.97 (dd, J = 9.2, 5.9 Hz, 1H), 7.47 (t, J = 9.0 Hz, 1H), 7.38 (d, J = 2.6 Hz, 1H), 7.22 (d, J = 2.6 Hz, 1H), 5.88-5.72 (m, 1H), 4.95-4.68 (m, 2H), 4.09 (s, 1H), 3.88 (s, 5H), 3.53 (s, 2H), 3.43 (s, 2H), 3.15- 3.08(m, 2H), 2.88-2.80 (m, 2H), 2.31-2.15 (m, 2H), 1.72- 1.68 (m, 3H), 1.63 (s, 4H), 1.36- 1.30 (m, 2H), 0.54 (p, J = 3.9 Hz, 1H), 0.30-0.19 (m, 1H) |
| 10 | 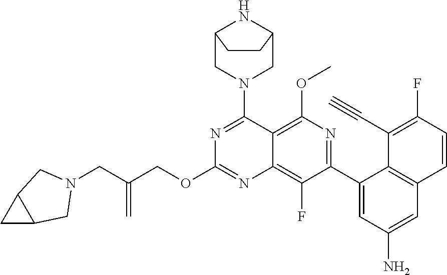 [SMILES: C#Cc1c(F)ccc2cc(N)cc(-c3nc(OC)c4c(N5CC6CCC(C5)N6)nc(OCC(=C)CN5CC6CC6C5)nc4c3F)c12] | 624.4 | δ 8.23 (s,1H), 7.77 (dd, J = 9.2, 5.9 Hz, 1H), 7.34 (t, J = 9.0 Hz, 1H), 7.06 (dd, J = 14.7, 2.4 Hz, 2H), 5.64 (s, 2H), 5.12-5.07 (m, 2H), 4.85 (d, J = 1.8 Hz, 2H), 4.13 (d, J = 12.6 Hz, 1H), 3.96 (d, J = 11.4 Hz, 1H), 3.89 (s, 3H), 3.82 (d, J = 1.1 Hz, 1H), 3.64 (d, J = 6.8 Hz, 2H), 3.50-3.38 (m, 2H), 2.89 (d, J = 8.6 Hz, 2H), 2.50 (s, 2H), 2.23 (dt, J = 8.6, 1.6 Hz, 2H), 1.67 (d, J = 10.4 Hz, 4H), 1.36- 1.31 (m, 2H), 0.58 (q, J = 3.8 Hz, 1H), 0.28 (td, J = 7.7, 3.8 Hz, 1H) |
| 11 | 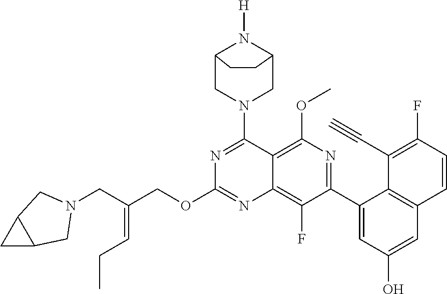 [SMILES: C#Cc1c(F)ccc2cc(O)cc(-c3nc(OC)c4c(N5CC6CCC(C5)N6)nc(OC/C(=C/CC)CN5CC6CC6C5)nc4c3F)c12] | 653.5 | δ 10.16 (s, 1H), 7.97 (dd, J = 9.2, 5.8 Hz, 1H), 7.47 (t, J = 9.0 Hz, 1H), 7.38 (d, J = 2.6 Hz, 1H), 7.22 (d, J = 2.5 Hz, 1H), 5.61 (t, J = 15 Hz, 1H), 4.85 (s, 2H), 4.10 (s, 1H), 3.88 (s, 5H), 3.46 (s, 2H), 3.04 (s, 2H), 2.82 (d, J = 9.0 Hz, 2H), 2.17 (dd, J = 16.9, 8.4 Hz, 5H), 2.01 (s, 1H), 1.60 (s, 4H), 1.31 (s, 2H), 1.24 (s, 4H), 0.93 (t, J = 7.4 Hz, 3H), 0.85 (d, J = 6.8 Hz, 1H), 0.54 (d, J = 3.2 Hz, 1H) |
| 12 | 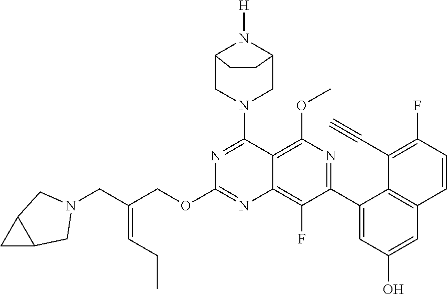 [SMILES: C#Cc1c(F)ccc2cc(O)cc(-c3nc(OC)c4c(N5CC6CCC(C5)N6)nc(OC/C(=C\CC)CN5CC6CC6C5)nc4c3F)c12] | 653.5 | δ 10.16 (s, 1H), 7.97 (dd, J = 9.2, 5.8 Hz, 1H), 7.47 (t, J = 9.0 Hz, 1H), 7.38 (d, J = 2.6 Hz, 1H), 7.22 (d, J = 2.5 Hz, 1H), 5.57 (t, J = 7.3 Hz, 1H), 4.85 (s, 2H), 4.10 (s, 1H), 3.88 (s, 5H), 3.46 (s, 2H), 3.04 (s, 2H), 2.82 (d, J = 9.0 Hz, 2H), 2.17 (dd, J = 16.9, 8.4 Hz, 5H), 2.01 (s, 1H), 1.60 (s, 4H), 1.31 (s, 2H), 1.24 (s, 4H), 0.93 (t, J = 7.4 Hz, 3H), 0.85 (d, J = 6.8 Hz, 1H), 0.54 (d, J = 3.2 Hz, 1H) |
| 13 | 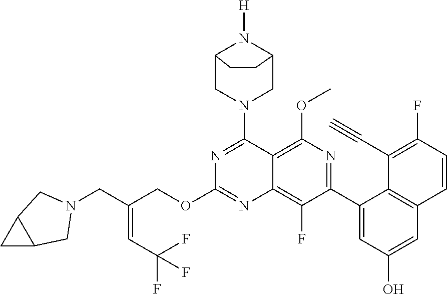 [SMILES: C#Cc1c(F)ccc2cc(O)cc(-c3nc(OC)c4c(N5CC6CCC(C5)N6)nc(OC/C(=C\C(F)(F)F)CN5CC6CC6C5)nc4c3F)c12] | 692.8 | δ 10.17 (s, 1H), 8.19 (s, 1H), 7.97 (dd, J = 9.2, 5.9 Hz, 1H), 7.47 (t, J = 9.0 Hz, 1H), 7.39 (d, J = 2.6 Hz, 1H), 7.22 (d, J = 2.6 Hz, 1H), 5.92 (q, J = 9.0 Hz, 1H), 5.05 (s, 2H), 4.18- 3.92 (m, 2H), 3.91-3.81 (m, 4H), 3.57-3.48 (m, 2H), 3.47- 3.39 (m, 2H), 3.38-3.35 (m, 2H), 2.90 (dd, J = 8.5, 2.5 Hz, 2H), 2.30 (d, J = 8.8 Hz, 2H), 1.67-1.51 (m, 4H), 1.39-1.31 (m, 2H), 0.56 (q, J = 3.8 Hz, 1H), 0.29 (td, J = 7.7, 3.9 Hz, 1H) |
| 14 | 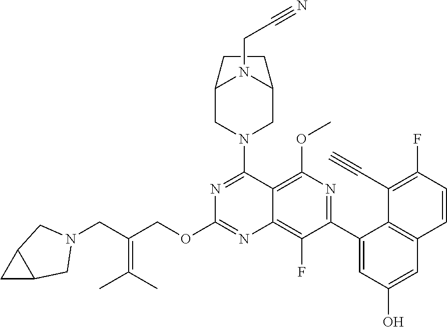 [SMILES: C#Cc1c(F)ccc2cc(O)cc(-c3nc(OC)c4c(N5CC6CCC(C5)N6CC#N)nc(OCC(CN5CC6CC6C5)=C(C)C)nc4c3F)c12] | 692.4 | δ 10.15 (s, 1H), 7.97 (dd, J = 9.2, 6.0 Hz, 1H), 7.47 (t, J = 9.1 Hz, 1H), 7.38 (d, J = 2.6 Hz, 1H), 7.22 (d, J = 2.5 Hz, 1H), 4.89 (s, 2H), 4.19-4.05 (m, 1H), 4.02-3.82 (m, 5H), 3.56 (s, 2H), 3.45-3.35 (m, 4H), 3.12 (s, 2H), 2.82 (dd, J = 8.6, 3.1 Hz, 2H), 2.26 (d, J = 8.4 Hz, 2H), 1.88-1.82 (m, 2H), 1.79 (s, 3H), 1.76 (s, 3H), 1.67- 1.51 (m, 2H), 1.29 (s, 2H), 0.55-0.47 (m, 1H), 0.29-0.15 (m, 1H) |
| 15 | 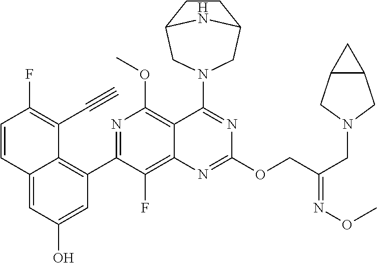 [SMILES: C#Cc1c(F)ccc2cc(O)cc(-c3nc(OC)c4c(N5CC6CCC(C5)N6)nc(OC/C(CN5CC6CC6C5)=N/OC)nc4c3F)c12] | 656.0 | δ 10.16 (s, 1H), 7.98 (dd, J = 9.2, 5.9 Hz, 1H), 7.47 (t, J = 9.0 Hz, 1H), 7.39 (d, J = 2.5 Hz, 1H), 7.21 (d, J = 2.6 Hz, 1H), 5.09 (s, 2H), 4.16-4.01 (m, 1H), 3.88 (s, 3H), 3.85 (d, J = 1.0 Hz, 1H), 3.81 (s, 3H), 3.47 (s, 2H), 3.41 (d, J = 12.8 Hz, 2H), 3.34 (s, 1H), 3.20-3.09 (m, 2H), 2.83-2.77 (m, 2H), 2.28 (dt, J = 8.1, 2.6 Hz, 2H), 1.66-1.46 (m, 4H), 1.28 (dt, J = 6.8, 3.0 Hz, 2H), 0.26 (q, J = 3.8 Hz, 1H), 0.16 (td, J = 7.6, 3.7 Hz, 1H) |
| 16 | 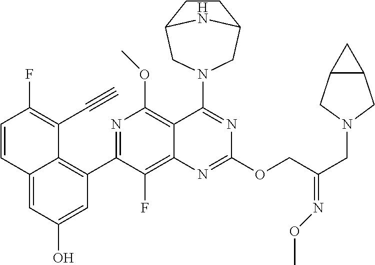 [SMILES: C#Cc1c(F)ccc2cc(O)cc(-c3nc(OC)c4c(N5CC6CCC(C5)N6)nc(OC/C(CN5CC6CC6C5)=N\OC)nc4c3F)c12] | 656.0 | δ 10.16 (s, 1H), 7.98 (dd, J = 9.2, 6.0 Hz, 1H), 7.47 (t, J = 9.0 Hz, 1H), 7.39 (d, J = 2.5 Hz, 1H), 7.22 (d, J = 2.5 Hz, 1H), 5.15-5.07 (m, 1H), 4.91 (s, 2H), 4.10 (s, 1H), 3.88 (s, 3H), 3.85 (t, J = 1.0 Hz, 1H), 3.78 (s, 3H), 3.49 (s, 2H), 3.39 (s, 3H), 2.87 (dd, J = 8.5, 3.9 Hz, 2H), 2.36-2.31 (m, 2H), 2.30-1.87 (m, 2H), 1.60 (s, 5H), 1.33- 1.22 (m, 3H), 0.45 (q, J = 3.8 Hz, 1H), 0.21 (td, J = 7.6, 3.8 Hz, 1H) |
| 17 | 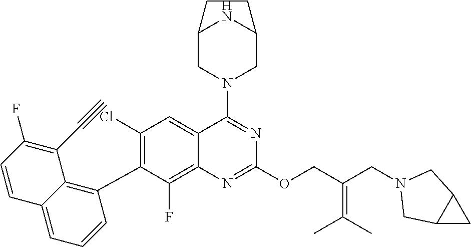 [SMILES: C#Cc1c(F)ccc2cccc(-c3c(Cl)cc4c(N5CC6CCC(C5)N6)nc(OCC(CN5CC6CC6C5)=C(C)C)nc4c3F)c12] | 656.3 | δ 10.29-10.06 (m, 1H), 8.01- 7.94 (m, 1H), 7.76 (d, J = 0.8 Hz, 1H), 7.51-7.43 (m, 1H), 7.39 (d, J = 2.6 Hz, 1H), 7.05 (d, J = 2.6 Hz, 1H), 4.90-4.82 (m, 2H), 4.28-4.20 (m, 2H), 3.93-3.88 (m, 1H), 3.56-3.43 (m, 4H), 3.12 (s, 2H), 2.81 (dd, J = 2.6, 8.4 Hz, 2H), 2.25 (d, J = 8.0 Hz, 2H), 1.79 (s, 3H), 1.75 (s, 3H), 1.66-1.62 (m, 2H), 1.33-1.18 (m, 4H), 0.54-0.46 (m, 1H), 0.20 (dt, J = 3.6, 7.6 Hz, 1H) |
| 18 | 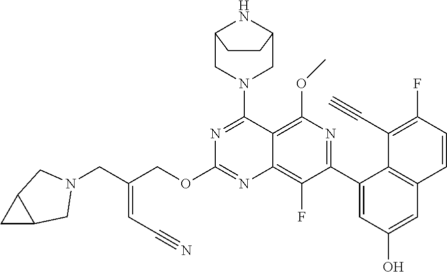 [SMILES: C#Cc1c(F)ccc2cc(O)cc(-c3nc(OC)c4c(N5CC6CCC(C5)N6)nc(OC/C(=C\C#N)CN5CC6CC6C5)nc4c3F)c12] | 649.8 | δ 10.18 (s, 1H), 7.97 (dd, J = 9.2, 5.9 Hz, 1H), 7.47 (t, J = 9.0 Hz, 1H), 7.39 (d, J = 2.8 Hz, 1H), 7.27-7.19 (m, 1H), 5.72- 5.03 (m, 3H), 4.08 (s, 1H), 3.89 (d, J = 6.0 Hz, 5H), 3.54 (s, 4H), 3.43 (s, 2H), 3.12 (s, 1H), 2.94 (d, J = 8.3 Hz, 2H), 2.38- 2.27 (m, 2H), 1.62 (s, 4H), 1.37 (d, J = 7.4 Hz, 2H), 0.58 (q, J = 3.9 Hz, 1H), 0.30 (td, J = 7.8, 4.0 Hz, 1H) |
| 19 | 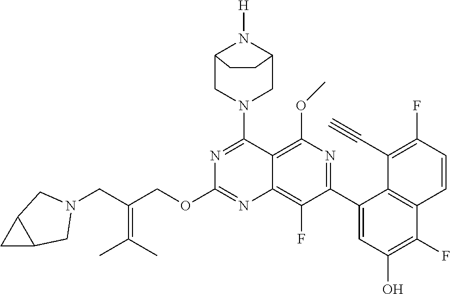 [SMILES: C#Cc1c(F)ccc2c(F)c(O)cc(-c3nc(OC)c4c(N5CC6CCC(C5)N6)nc(OCC(CN5CC6CC6C5)=C(C)C)nc4c3F)c12] | 671.3 | δ 10.55 (s, 1H), 8.13 (dd, J = 9.3, 5.7 Hz, 1H), 7.61 (t, J = 9.1 Hz, 1H), 7.41 (d, J = 8.6 Hz, 1H), 4.89 (s, 2H), 4.21-4.05 (m, 1H), 4.03 (s, 1H), 4.01- 3.93 (m, 1H), 3.89 (s, 3H), 3.71 (s, 2H), 3.44 (t, J = 14.3 Hz, 2H), 3.13 (s, 2H), 2.89-2.75 (m, 2H), 2.27 (d, J = 8.5 Hz, 2H), 1.79 (s, 3H), 1.76 (s, 3H), 1.74-1.62 (m, 4H), 1.30 (s, 2H), 0.57-0.47 (m, 1H), 0.26- 0.18 (m, 1H) |

### Biological Assays/Testing

### Cell Lines

The following cancer cell lines were employed: AGS gastric carcinoma [heterozygous G121D](ATCC, CRL-1739); A-427 lung carcinoma [heterozygous G12D](ATCC, HTB-53); ASPC1 pancreatic adenocarcinoina [homozygous G121D](ATCC, CRL-1682) and SW1990 pancreatic adenocarcinoma [homozygous G12D](ATCC CRL-2172). Cell lines were cultured essentially according to ATCC recommendations.

### KRAS(G12D)/SOS1 Homogeneous Time-Resolved Fluorescence (HTRF) Assay

Binding of test compounds to KRAS(G12D) target protein, which in turn blocks KRAS(G12D) interaction with the SOS1 protein, was measured in the absences of GTP by homogeneous time-resolved fluorescence using the KRAS-G12D/SOS1 Binding Assay Kit (Cisbio, 63ADK000CB17PEH), following the manufacturer's instructions, except as noted. 3-fold serial dilutions of each test compound were prepared ranging from 20 M to 1.02 nM. The test compound was mixed and incubated with reaction components, incubated in a sealed plate at 4° C. for 3 hr and fluorescence was measured using a PerkinElmer Envision plate reader. The KRAS(G12D)-SOS1 IC50 values (the concentration of 50% of the maximal inhibition) were calculated using GraphPad Prism 7 software.

### Cancer Cell Line Proliferation (CellTiter-Glo® Assays)

AGS, SW1990, and GP2D cells were plated in 96-well tissue culture plates at 4,000 cells/well and incubated at 37° C./5% CO2 for 72 hr in 100 μl of media. 3-fold serial dilutions of each test compound were prepared ranging from 20 M to 1.02 nM. Each cell line was then treated with test compounds at various concentrations with a final concentration of 0.5% DMSO/well, and then incubated at 37° C./5% CO2 for 72 hr. 100 μl of CellTiter-Glo® Reagent (Promega Corporation, Madison, WI) was added to each well and processed according manufacturer's protocol. Results were analyzed and IC50 values were calculated in GraphPad 7 software.

Results are listed in Table 2. KRAS(G12D)/SOS1 HTRF assay: A. IC50<100 nM; B. IC50=100-1000 nM; C. IC50>1000 nM; AGS proliferation assay: A. EC50<100 nM; B. EC50=100-1000 nM; C. EC50>1000 nM; GP2D proliferation assay: A. EC50<100 nM; B. EC50=100-1000 nM; C. EC50>1000 nM; SW1990 proliferation assay: A. EC50<100 nM; B. EC50=100-1000 nM; C. EC50>1000 nM.

TABLE 2
Biochemical and Cell-based Assays of Compounds

| | |
|---|---|
| | CTG(IC50) |
| HTRF of KRAS | ABS-72H(nM) |

| Ex. | G12D-SOS1(nM) | AGS | GP2D | SW1990 |
|---|---|---|---|---|
| 1 | A | A | A | A |
| 2 | A | A | A | A |
| 3 | A | A | A | A |
| 4 | A | A | A | A |
| 5 | A | A | A | A |
| 6 | | A | A | A |
| 7 | | B | B | |
| 8 | | B | B | |
| 9 | | A | A | |
| 10 | | B | A | |
| 11 | | A | A | |
| 12 | | B | A | |
| 13 | | B | B | |
| 14 | | A | A | |
| 15 | | A | A | |
| 16 | | A | A | |
| 17 | | B | B | |
| 18 | | B | | |
| 19 | | B | A | |

### Oral Bioavailability

CD-1 mice were randomly assigned to 6 groups with 3 male mice in each compound group. Compounds were orally administered (P0) in a single dose to each mouse in its group. Blood samples were taken within 72 hours. Bioavailability (F %) was determined and by liquid chromatography-mass spectrometry (LC-MS/MS). The mean oral % F is provided in Table 3.

TABLE 3

| Ex. | % F |
|---|---|
| 1 | 10.6 |
| 2 | 12.1 |
| 4 | 1.7 |
| 5 | 7.7 |

Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

All patents and publications mentioned in this specification are herein incorporated by reference to the same extent as if each independent patent and publication was specifically and individually indicated to be incorporated by reference.

1. A compound of the Formula I: 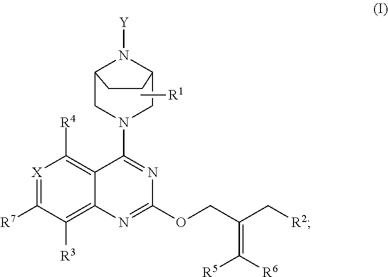
or a pharmaceutically acceptable salt thereof, wherein
Y is hydrogen or —C(O)OCHR^{a}OC(O)R^{b};
X is —CR^{4a} or N;
R¹ is hydrogen, halo, OH, (C₁-C₄)alkyl, (C₁-C₄)haloalkyl, (C₁-C₄)hydroxyalkyl, —CHO, —C(O)OR^{b}, —C(O)ONR^{a}R^{b} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;
R² is a heterocyclyl optionally substituted with 1 to 3 groups selected from R¹;
R³ is selected from hydrogen, halo, (C₁-C₄)alkyl, cyano, and (C₃-C₆)cycloalkyl optionally substituted with 1 to 3 groups selected from halo, (C₁-C₄)alkyl, (C₁-C₄)alkoxy, halo(C₁-C₄)alkoxy, and cyano;
R⁴ is selected from hydrogen, (C₁-C₄)alkyl, (C₁-C₄)haloalkyl, (C₁-C₄)alkoxy, deuterated (C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
R^{4a} is selected from hydrogen, halo, (C₁-C₄)alkyl, (C₁-C₄)haloalkyl, C₁-C₄)haloalkoxy, (C₂-C₄)alkynyl, (C₂-C₄)alkenyl, (C₁-C₄)alkoxy, halo(C₁-C₄)alkoxy, (C₃-C₆)cycloalkyl, —O(C₃-C₆)cycloalkyl, cyano, NH₂, —NH(C₁-C₄)alkyl, and —N[(C₁-C₄)alkyl]₂;
or R⁴ and R^{4a} are taken together with the atoms to which they are respectively attached form a 5- to 6-membered heterocyclyl ring or a 5- to 6-membered heteroaryl ring, each optionally substituted with 1 or 2 groups selected from halo, (C₁-C₄)alkyl, (C₁-C₄)haloalkyl, (C₁-C₄)alkoxy, and (C₁-C₄)haloalkoxy;
R⁵ and R⁶ are each independently selected from hydrogen, halo, (C₁-C₄)alkyl, (C₁-C₄)haloalkyl, (C₁-C₄)alkoxy, deuterated (C₁-C₄)alkoxy, and (C₁-C₄)haloalkoxy; or R⁵ and R⁶ are taken together with the carbon they are attached to form (C₃-C₆)cycloalkyl or 4- to 8-membered monocyclic heterocyclyl, wherein said (C₃-C₆)cycloalkyl and said 4- to 8-membered monocyclic heterocyclyl are optionally substituted with 1 to 3 groups selected from halo, (C₁-C₄)alkyl, (C₁-C₄)alkoxy, halo(C₁-C₄)alkoxy, and cyano;
R⁷ is selected from aryl or 5- to 10-membered heteroaryl, wherein said aryl and 5- to 10-membered heteroaryl are each optionally substituted with one or more groups selected from R^{7a};
R^{7a} is selected from halo, (C₁-C₄)alkyl, halo(C₁-C₄)alkyl, (C₁-C₄)alkoxy, halo(C₁-C₄)alkoxy, (C₂-C₄)alkenyl, (C₂-C₄)alkynyl, cyano, OH, NH₂, —NH(C₁-C₄)alkyl, —N[(C₁-C₄)alkyl]₂, (C₃-C₆)cycloalkyl, and —O(C₃-C₆)cycloalkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
R^{a} and R^{b} are each independently selected from hydrogen and (C₁-C₄)alkyl; and
R^{c} is selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO₂.

2. The compound of claim 1, or a pharmaceutically acceptable salt thereof, wherein Y is hydrogen.

3. The compound of claim 1 or 2, or a pharmaceutically acceptable salt thereof, wherein R¹ is hydrogen.

4. The compound of any one of claims 1 to 3, or a pharmaceutically acceptable salt thereof, wherein R⁴ is hydrogen or (C₁-C₄)alkoxy.

5. The compound of any one of claims 1 to 4, or a pharmaceutically acceptable salt thereof, wherein R⁴ is hydrogen or OCH₃.

6. The compound of any one of claims 1 to 5, or a pharmaceutically acceptable salt thereof, wherein X is N.

7. The compound of any one of claims 1 to 3, or a pharmaceutically acceptable salt thereof, wherein X is —CR^{4a} and R⁴ and R^{4a} are taken together with the atoms to which they are respectively attached form a 5-membered heterocyclyl ring.

8. The compound of any one of claims 1 to 3, or a pharmaceutically acceptable salt thereof, wherein X is —CR^{4a} and R⁴ and R^{4a} are taken together with the atoms to which they are respectively attached form a dihydrofuranyl or a dihydropyrazolyl ring.

9. The compound of any one of claims 1 to 8, or a pharmaceutically acceptable salt thereof, wherein R³ is halo.

10. The compound of any one of claims 1 to 9, or a pharmaceutically acceptable salt thereof, wherein R³ is fluoro.

11. The compound of any one of claims 1 to 10, or a pharmaceutically acceptable salt thereof, wherein R⁵ and R⁶ are each independently selected from hydrogen, halo, (C₁-C₄)alkyl, and (C₁-C₄)haloalkyl.

12. The compound of any one of claims 1 to 11, or a pharmaceutically acceptable salt thereof, wherein the combination of R⁵ and R⁶ correspond to structures selected from

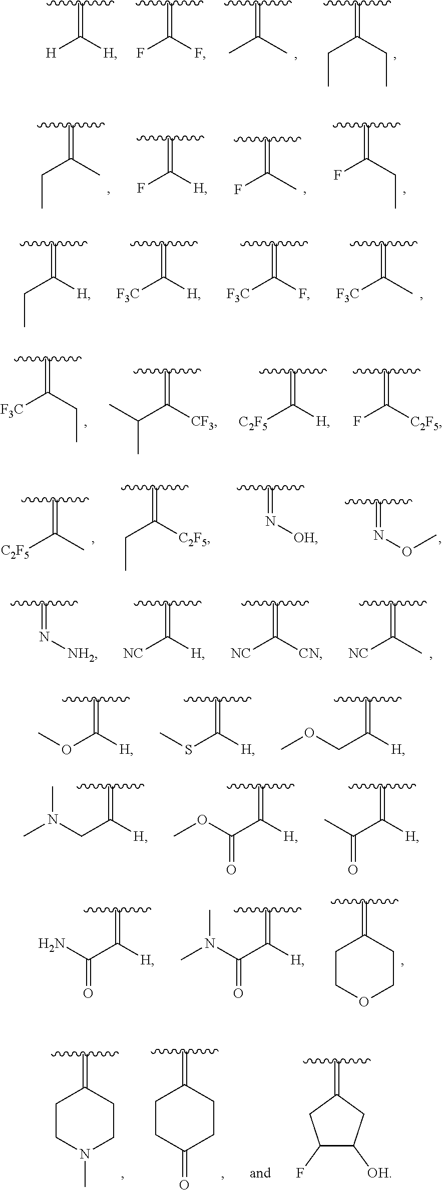

13. The compound of any one of claims 1 to 10, or a pharmaceutically acceptable salt thereof, wherein R⁵ and R⁶ are taken together with the carbon they are attached to form (C₃-C₆)cycloalkyl or 4- to 6-membered monocyclic heterocyclyl, each optionally substituted with 1 to 3 groups selected from halo, (C₁-C₄)alkyl, (C₁-C₄)alkoxy, halo(C₁-C₄)alkoxy, and cyano.

14. The compound of any one of claims 1 to 10, or a pharmaceutically acceptable salt thereof, wherein R⁵ and R⁶ are taken together with the carbon they are attached to form cyclopropyl, cyclobutyl, cyclopentyl, cyclohexyl, oxetanyl, azetidinyl, tetrahydropyranyl, or piperidinyl, each optionally substituted with 1 to 3 (C₁-C₄)alkyl.

15. The compound of any one of claims 1 to 14, or a pharmaceutically acceptable salt thereof, wherein R⁷ is selected from phenyl, naphthalenyl, 6-membered heteroaryl, 9-membered heteroaryl, or 10-membered heteroaryl, each of which are optionally substituted with one or more groups selected from R^{7a}.

16. The compound of any one of claims 1 to 15, or a pharmaceutically acceptable salt thereof, wherein R⁷ is selected from phenyl, naphthalenyl, pyridinyl, benzothiazolyl, or isoquinolinyl, each of which are optionally substituted with one or more groups selected from R^{7a}.

17. The compound of any one of claims 1 to 16, or a pharmaceutically acceptable salt thereof, wherein R^{7a} is selected from halo, (C₁-C₄)alkyl, halo(C₁-C₄)alkyl, halo(C₁-C₄)alkoxy, (C₂-C₄)alkynyl, cyano, OH, NH₂, and (C₃-C₆)cycloalkyl.

18. The compound of any one of claims 1 to 17, or a pharmaceutically acceptable salt thereof, wherein R^{7a} is selected from fluoro, chloro, ethyl, ethynyl, CF₃, C₂F₅, cyano, OCF₃, OH, NH₂, and cyclopropyl.

19. The compound of any one of claims 1 to 18, or a pharmaceutically acceptable salt thereof, wherein R⁷ is selected from

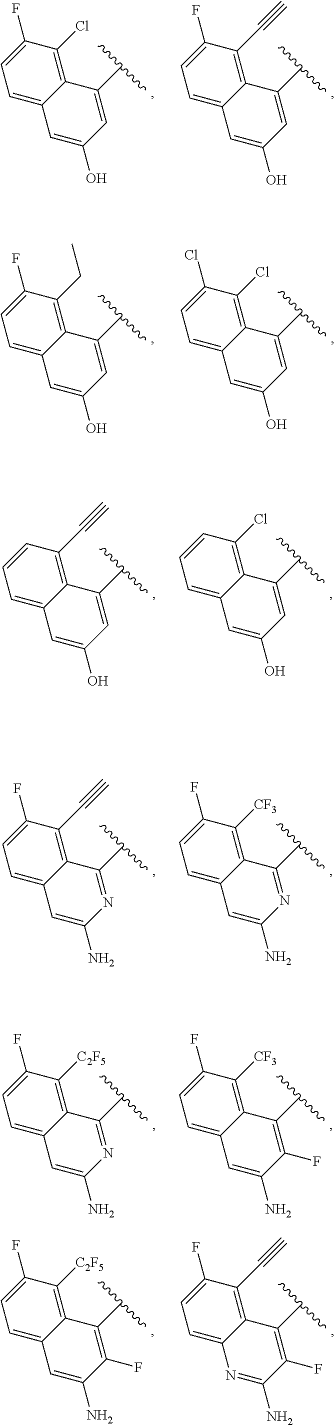

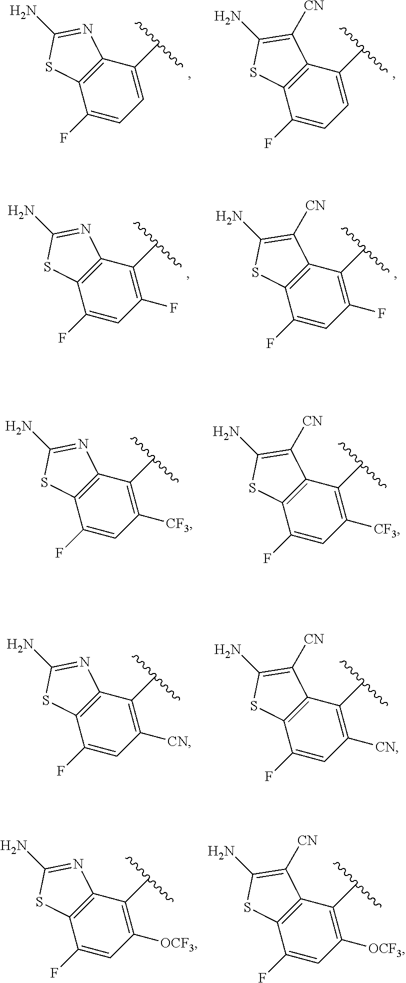

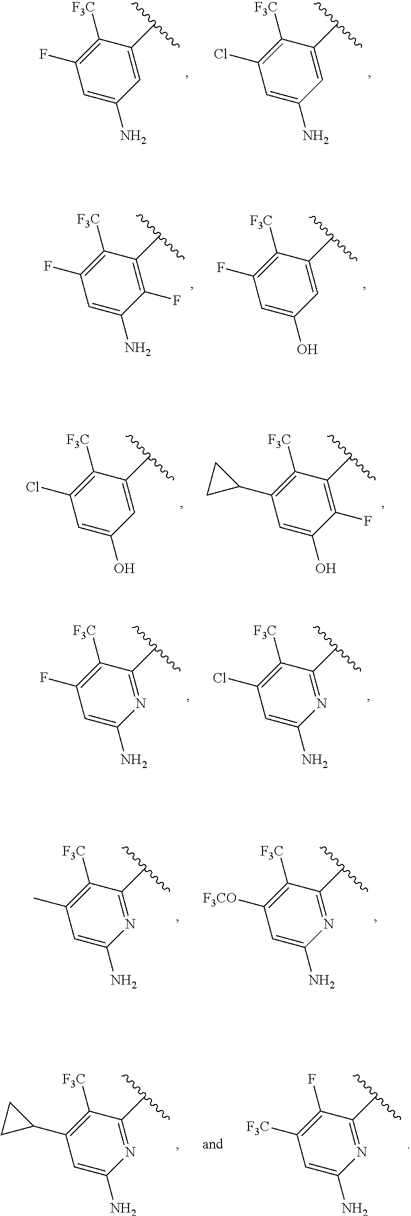

20. The compound of any one of claims 1 to 19, or a pharmaceutically acceptable salt thereof, wherein R² is a 4- to 6-membered monocyclic heterocyclyl or a 6- to 10-membered bicyclic heterocyclyl, each optionally substituted with 1 to 3 groups selected from R^{c}.

21. The compound of any one of claims 1 to 20, or a pharmaceutically acceptable salt thereof, wherein R² is pyrrolidinyl, 3-azabicyclo[3.1.0]hexanyl, octahydrocyclopenta[c]pyrrolyl, 6-oxa-2-azaspiro[4.5]decanyl, 5-azaspiro[2.4]heptanyl, 2-azaspiro[3.3]heptanyl, 2-oxa-6-azaspiro[3.3]heptanyl, 6-oxa-2-azaspiro[3.4]octanyl, 8-oxa-2-azaspiro[4.5]decanyl, azetidinyl, piperidinyl, morpholinyl, piperizinyl, 6-azaspiro[2.5]octanyl, 8-azabicyclo[3.2.1]octanyl, 3-oxa-8-azabicyclo[3.2.1]octanyl, 5,6,7,8-tetrahydro-1,7-naphthyridinyl, 3-oxa-8-azabicyclo[3.2.1]octanyl, 4,5,6,7-tetrahydrothieno[2,3-c]pyridinyl, 2-oxa-7-azaspiro[3.5]nonanyl, 2-oxa-7-azaspiro[4.4]nonanyl, 1-oxa-7-azaspiro[4.4]nonanyl, isoindolinyl, and 6,7-dihydro-5H-pyrrolo[3,4-b]pyridinyl, each optionally substituted with 1 to 3 groups selected from R^{c}.

22. The compound of any one of claims 1 to 21, or a pharmaceutically acceptable salt thereof, wherein R^{c} is selected from is selected from halo, (C₁-C₄)haloalkyl, (C₁-C₄)haloalkoxy, cyano, and —C(O)OR^{a}.

23. The compound of any one of claims 1 to 22, or a pharmaceutically acceptable salt thereof, wherein R² is selected from

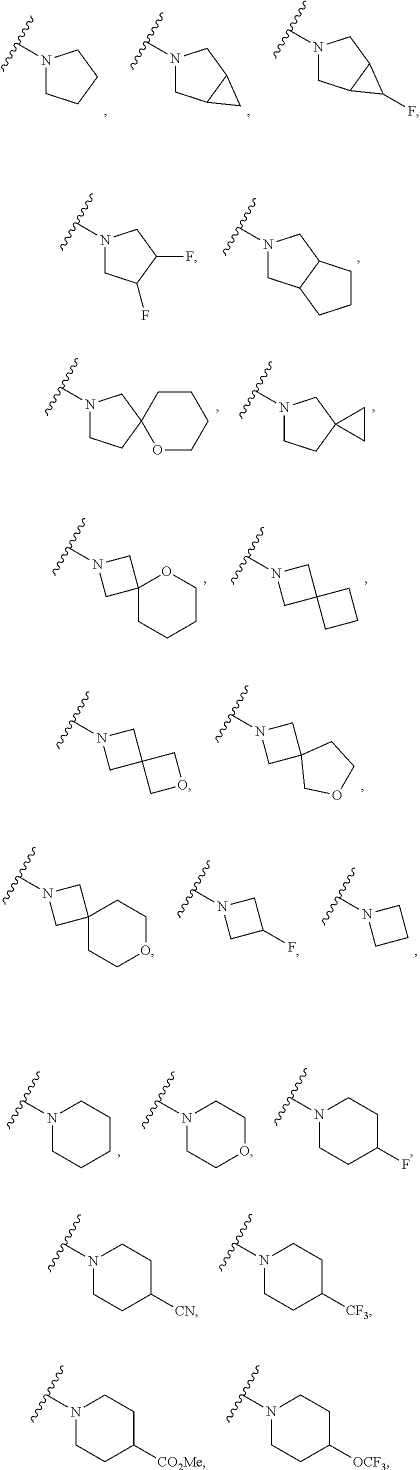

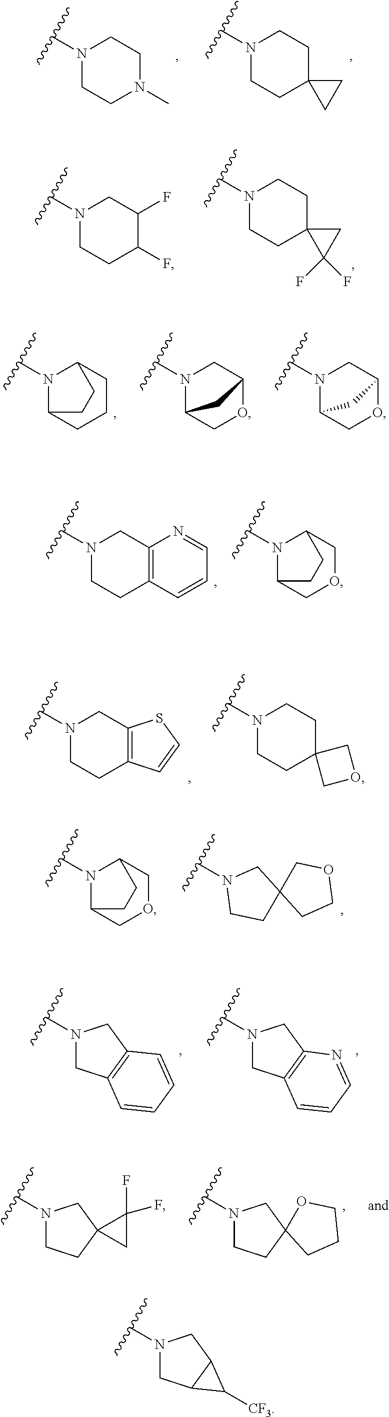

24. A pharmaceutical composition comprising a compound of any one of claims 1 to 23, or a pharmaceutically acceptable salt thereof; and a pharmaceutically acceptable carrier.

25. A method for treating cancer in a subject comprising administering to the subject and effective amount of a compound of any one of claims 1 to 23, or a pharmaceutically acceptable salt thereof, or the pharmaceutically acceptable composition of claim 24.
